(19) United States
(12) Patent Application Publication
(10) Pub. No.: US 2022/0170002 A1
(43) Pub. Date: Jun. 2, 2022

(54) AMYLASE COMPOSITION

(71) Applicant: NAGASE CHEMTEX CORPORATION, Osaka-shi, Osaka (JP)

(72) Inventor: Manabu IIJIMA, Kyoto (JP)

(21) Appl. No.: 17/436,945

(22) PCT Filed: Mar. 11, 2020
(86) PCT No.: PCT/JP2020/010428

§ 371 Date: Sep. 7, 2021

(30) Foreign Application Priority Data
Mar. 11, 2019 (JP) 2019-043746
Feb. 19, 2020 (JP) PCT/JP2020/006424

(51) Int. Cl. C12N 9/26; A23L 29/00; A23L 29/30

(52) U.S. Cl. CPC C12N 9/2425; C12Y 302/01002; A23V 2002/00; A23L 29/35; A23L 29/37; A23L 29/06

(57) ABSTRACT

The present invention provides an amylase composition which enables long-term stable storage of amylase without using chemicals that may affect the human body in food use. The present invention relates to an amylase composition containing a saccharide containing glucose as a constituent unit, a polyhydric alcohol, and an amylase.

### TECHNICAL FIELD

The present invention relates to an amylase composition.

### BACKGROUND ART

β-Amylase is used to produce maltose or starch syrup and is also used as preparations for preventing staling of mochi (rice cakes), mochi confectioneries, and the like. β-Amylase includes: those derived from plants such as soybean, barley, wheat, and sweet potato and those derived from microorganisms. Soybean-derived β-amylase has higher heat resistance than liquid barley-derived β-amylase preparations and shows activity even at a high temperature of about 60° C. Thus, it is expected that soybean-derived β-amylase can be used under wider conditions than β-amylase derived from sources other than soybean. Moreover, soybean-derived β-amylase, when added in an amount that is half the amount of barley-derived β-amylase, produces an equivalent saccharification effect. Thus, it is expected to be able to reduce the amount of enzyme used and the cost.

Liquid enzyme preparations generally contain stabilizers for maintenance of enzyme activity and preservation. To achieve a high preservation effect, chemicals such as benzoic acid, parabens, and sorbic acid may be added. These chemicals however correspond to designated additives whose use in food is restricted, and tend to be shunned in the market due to growing concern about food safety.

In order to improve the storage stability of products, the growth of microorganisms in enzyme preparations can be reduced by adding a salt such as sodium chloride to the enzyme preparations to reduce the water activity or increase the osmotic pressure. However, when β-amylase preparations containing salts are used to produce maltose, the salts may adversely affect the purification process using an ion exchange resin, particularly the decolorization and desalination step.

Patent Literature 1 discloses the addition of a polyhydric alcohol and a saccharide to prevent autolysis by protease and precipitation in an aqueous solvent. However, no method has been known to improve the long-term storage stability of amylase.

### CITATION LIST

### Patent Literature

Patent Literature 1: JP 2017-029129 A

### SUMMARY OF INVENTION

### Technical Problem

An object of the present invention is to provide an amylase composition which enables long-term stable storage of amylase without using chemicals that may affect the human body in food use.

### Solution to Problem

The present inventor has made extensive studies and has found that the combined use of a polyhydric alcohol and a saccharide containing glucose as a constituent unit can achieve long-term storage stability of amylase. This finding has led to the completion of the present invention.

That is, the present invention relates to an amylase composition, containing:

a saccharide containing glucose as a constituent unit;

a polyhydric alcohol; and

an amylase.

Preferably, a combined amount of the saccharide containing glucose as a constituent unit and the polyhydric alcohol is 70% by weight or less.

Preferably, the polyhydric alcohol is selected from the group consisting of glycerol, sorbitol, and propylene glycol.

Preferably, the saccharide is at least one selected from the group consisting of sucrose, trehalose, dextrin, maltose, and maltitol.

Preferably, the amylase is a β-amylase.

The present invention also relates to a food additive, containing the above amylase composition.

The present invention also relates to a food, containing the above food additive.

The present invention also relates to a method of stabilizing an amylase, the method including

mixing an amylase, a polyhydric alcohol, and a saccharide containing a glucose unit.

### Advantageous Effects of Invention

The amylase composition of the present invention is free from chemicals that may affect the human body in food use and improves the long-term storage stability of amylase. Moreover, the amylase composition of the present invention, which is free from salts as essential components, does not impose a burden on the production process of maltose.

### BRIEF DESCRIPTION OF DRAWINGS

FIG. 1 shows the relative activities (residual activities) of the amylase compositions of Comparative Examples 1 to 4.

FIG. 2 shows the relative activities (residual activities) of the amylase compositions of Examples 1 to 3 and Comparative Examples 5 and 6.

FIG. 3 shows the relative activities (residual activities) of the amylase compositions of Examples 4 to 7 and Comparative Example 7.

FIG. 4 shows the relative activities (residual activities) of the amylase compositions of Examples 8 to 12 and Comparative Examples 7 and 8.

FIG. 5 shows the relative activities (residual activities) of the amylase compositions of Example 13 and Comparative Examples 8 to 13.

### DESCRIPTION OF EMBODIMENTS

### <<Amylase Composition>>

The amylase composition of the present invention contains a saccharide containing glucose as a constituent unit, a polyhydric alcohol, and an amylase.

### <Amylase>

Amylases are enzymes that cleave α-1,4-bonds of starch, glycogen, or the like to form maltose. Amylases are classified into α-amylase, β-amylase, glucoamylase, isoamylase, maltogenic amylase, and other amylases. The amylase in the present invention is preferably, but not limited to, a β-amylase that can be used to produce a preparation having high heat resistance.

The amylase in the present invention may be derived from any source, including plants, animals, and microorganisms. Preferred among these amylases are amylases produced by microorganisms such as Aspergillus, Bacillus, and Streptomyces, and amylases derived from soybean, barley, wheat, or sweet potato, which have been eaten and can be easily applied to foods, with soybean-derived β-amylase being more preferred. The soybean-derived β-amylase, which has excellent heat resistance and reactivity, is expected to be useful in a wide range of applications as compared to those derived from sources other than soybean such as barley-derived β-amylase.

The amylase may be either one extracted from a source plant, animal, or microorganism or one mass-produced by a gene recombination technique. The amylase may be a wild-type amylase or a variant amylase.

The amylase may be obtained as follows. When an amylase accumulates in the cells of a source organism, the tissue and cells may be homogenized and subjected to centrifugation or other treatment to obtain a cell-free extract which may be used as the amylase. Alternatively, an amylase may be purified from the cell-free extract as a starting material by a general protein purification method such as salting-out or various chromatographic techniques (e.g., ion exchange chromatography, gel filtration chromatography, hydrophobic chromatography, affinity chromatography). When an amylase produced by a microorganism is secreted extracellularly, the amylase may be purified from the culture medium. The amylase used in the present invention is not limited to a pure product, and may be present in a crude product such as a plant extract (e.g., soybean whey) or a cell-free extract of a microorganism.

To maintain a high amylase activity, the amount of the amylase in the amylase composition is preferably 30% by weight or more, more preferably 50% by weight or more, still more preferably 70% by weight or more, further more preferably 80% by weight or more, particularly preferably 90% by weight or more.

### <Polyhydric Alcohol>

The polyhydric alcohol may be any alcohol that contains two or more hydroxy groups and that can reduce the water activity. Specific examples of the polyhydric alcohol include glycerol, sorbitol, propylene glycol, polyvinyl alcohol, pentaerythritol, ethylene glycol, diethylene glycol, triethylene glycol, and polyethylene glycol. Preferred among these is glycerol.

The amount of the polyhydric alcohol in the amylase composition is preferably 30 to 60% by weight, more preferably 30 to 55% by weight. With an amount less than 30% by weight, the amylase tends not to be sufficiently stabilized, while with an amount more than 60% by weight, the amylase activity in the composition will be excessively diluted.

### <Saccharide Containing Glucose as Constituent Unit>

The saccharide may be any mono-, di-, tri- or higher polysaccharide containing glucose. The disaccharide or polysaccharide may be any substance in which monosaccharide molecules are polymerized through glycosidic bonds and which contains glucose as one of the monosaccharide molecules. In the disaccharide or polysaccharide, the glucose is preferably linked via an α-1,4-bond. Specific examples of the saccharide containing glucose as a constituent unit include dextrin, maltose, maltitol, sucrose, lactose, trehalose, and cellobiose. Preferred among these are dextrin, sucrose, trehalose, and maltitol which do not contain or contain a small proportion of reducing groups that can cause browning to deteriorate product quality during storage.

The amount of the saccharide containing glucose as a constituent unit in the amylase composition is preferably 1 to 20% by weight, more preferably 2 to 15% by weight. With an amount less than 1% by weight, the amylase tends not to be sufficiently stabilized, while with an amount more than 20% by weight, the amylase activity in the composition cannot be enhanced.

Moreover, the combined amount of the saccharide containing glucose as a constituent unit and the polyhydric alcohol in the amylase composition is preferably 70% by weight or less, more preferably 60% by weight or less, still more preferably 50% by weight or less, further more preferably 45% by weight or less. With a combined amount more than 70% by weight, the concentration of the amylase in the amylase composition will be reduced. In order to enhance the concentration of the amylase in the amylase composition, the combined amount of the saccharide containing glucose as a constituent unit and the polyhydric alcohol is preferably smaller, but the lower limit of the combined amount is generally 30% by weight.

The amylase composition may be in any form and may be either liquid or solid. The liquid form may be, for example, an aqueous solution, a suspension, or a slurry. The solid form may be, for example, powder, granules, or a tablet. Among these, the liquid form is preferred in terms of cost and handleability. In the case of an amylase composition in the liquid form, it is conventionally considered difficult to maintain the enzyme activity of the amylase and reduce microbial contamination and other problems. In contrast, it is possible for the amylase composition of the present invention to stably maintain the amylase activity.

When the amylase composition is in the liquid form, the pH of the amylase composition is preferably 4 to 9, more preferably 4.5 to 7, still more preferably 5 to 6. With a pH less than 4, precipitates tend to occur. With a pH more than 9, the activity of the amylase tends to be impaired. The pH of the amylase composition can be controlled with acid (e.g., hydrochloric acid or sulfuric acid) or base (e.g., sodium hydroxide or potassium hydroxide).

The amylase composition may have any enzyme activity. The enzyme activity is generally preferably 1,000 to 1,000,000 units/g or less. Here, one unit of the enzyme activity is defined as the amount of enzyme that produces 1 mg of maltose per hour under conditions including pH 5.5 and 60° C.

The amylase composition of the present invention containing a polyhydric alcohol and a saccharide containing glucose as a constituent unit can stably maintain the activity of the amylase for a long period of time. Even after storage at 40° C. for three months, the amylase composition preferably maintains 70% or higher, preferably 80% or higher activity compared to before storage. The activity can be evaluated based on the above-described enzyme activity.

The amylase composition of the present invention makes it possible to prevent the growth of viable bacteria in the composition. Preferably, viable bacteria will not grow to 10,000 or more cells even after storage at 40° C. for three months.

### <Method of Preparing Composition>

The amylase composition can be prepared by mixing components in any order. After the components are mixed, the mixture may be subjected to filtration or bacteria elimination by contact with a porous material or passing through a filter.

The amylase composition may contain any component in addition to the saccharide containing glucose as a constituent unit, the polyhydric alcohol, and the amylase.

### <<Food Additive>>

The food additive of the present invention is characterized by containing the above-described amylase composition. The food additive may contain additional components acceptable in food in addition to the amylase composition. Examples of such additional components include excipients, pH adjusters, enzymes, thickening polysaccharides, emulsifiers, mixtures of emulsifiers and polyphosphates, dairy products, extracts, sweeteners, fermented seasonings, eggs, inorganic salts, preservatives, organic acids, metals, and filter aids. These components may be present in any amount, and one skilled in the art may select any amount of the components.

Examples of the pH adjusters include ascorbic acid, acetic acid, dehydroacetic acid, lactic acid, citric acid, gluconic acid, succinic acid, tartaric acid, fumaric acid, malic acid, and adipic acid, and sodium (Na), calcium (Ca), or potassium (K) salts of these organic acids; and carbonic acid, phosphoric acid, and pyrophosphoric acid, and Na or K salts of these inorganic acids.

Examples of the enzymes include carbohydrate-related enzymes such as α-amylase, glucoamylase, pullulanase, isoamylase, maltotriohydrolase, cyclodextrin glucanotransferase, transglucosidase, dextranase, glucose isomerase, cellulase, xylanase, hemicellulase, mannanase, pectinase, pectin methylesterase, invertase, lactase, inulinase, α-galactosidase, chitinase, chitosanase, and alginate lyase; protein or amino acid-related enzymes such as protease, peptidase, collagenase, and glutaminase; lipid-related enzymes such as lipase, phospholipase, and esterase; and other enzymes such as catalase, glucose oxidase, urease, tannase, and deaminase.

Examples of the thickening polysaccharides include modified starches, gums, alginic acid, alginic acid derivatives, pectin, carrageenan, curdlan, pullulan, gelatin, cellulose derivatives, agar, tamarind, psyllium, and glucomannan.

Examples of the emulsifiers include glycerol fatty acid esters, polyglycerol fatty acid esters, sucrose fatty acid esters, propylene glycol fatty acid esters, sorbitan fatty acid esters, lecithin, enzymatically decomposed lecithin, and saponin.

Examples of the dairy products include milk, skim milk powder, whole milk powder, whey powder, casein, cheese, yogurt, condensed milk, fermented milk, and cream.

Examples of the extracts include yeast extract and malt extract.

Examples of the sweeteners include stevia, aspartame, glycyrrhizin, acesulfame potassium, sucralose, and neotame.

Examples of the inorganic salts include sodium chloride, ammonium sulfate, sodium sulfate, calcium chloride, and polyphosphates.

Examples of the preservatives include propionic acid, propionates, sulfites, benzoates, sorbic acid, sorbates, milt protein, polylysine, glycine, and acetates. Examples of the salts include sodium (Na), calcium (Ca), and potassium (K) salts.

### <<Food>>

The food of the present invention is characterized by containing the above-described food additive. The food of the present invention is preferably a processed grain food. Examples of the grain include rice, azuki beans, rye, barley, buckwheat, wheat, sweet potatoes, potatoes, tapioca, arrowroot, corn, Chinese yam, taro, lily roots, lotus roots, lentils, chickpeas, common beans, peas, broad beans, peanuts, Shirohanamame (Phaseolus coccineus), soybeans, and sweetened green peas. Preferred among these are processed rice or wheat foods. Examples of the processed rice foods include cooked rice, okowa (steamed rice), chimaki (zongzi), onigiri (rice balls), sushi, fried rice, and mochi (rice cakes). Examples of the processed wheat foods include breads, cakes, confectioneries, and noodles.

Processed grain foods tend to harden due to starch staling during long-term storage, resulting in deterioration of texture. The starch staling is caused by partial retrogradation of pregelatinized starch. Amylase cleaves maltose, a glucose dimer, from the end of the sugar chain to shorten the sugar chain and prevents partial retrogradation to prevent starch staling. In the food of the present invention containing the amylase composition, starch staling is reduced.

In the production of the food of the present invention, the food additive of the present invention may be incorporated at any time. The food additive may be added to and/or mixed with ingredients before the production of the food, or alternatively, the food additive may be incorporated during the production of the food to allow the amylase to act.

When the method for the production of the food includes a heating step, the food additive of the present invention may be added before or after the heating step. When the food additive is added before the heating step, the amylase is allowed to act until the heating step, and the anti-staling effect is also maintained for a long time after the heating. When the food additive is added after the heating step, the anti-staling effect is maintained for a long time after the production of the food.

The food additive of the present invention is preferably added at a temperature of 4 to 70° C., more preferably of 25 to 65° C., still more preferably of 50 to 60° C. When soybean-derived β-amylase, which has high heat resistance, is used, it may be allowed to act at a high temperature of 60° C. or higher, for example. Moreover, in order to reduce the amount of the amylase added while achieving the anti-staling effect, the food additive of the present invention is preferably added after the food is at a low temperature.

The food of the present invention may be stored after the incorporation of the food additive of the present invention. The storage temperature is preferably −80 to 30° C. In particular, the storage temperature during refrigeration is preferably −20 to 0° C., more preferably −10 to −4° C. The storage temperature during refrigeration is preferably 0 to 10° C., more preferably 0 to 4° C. The storage temperature during storage at room temperature is preferably 15 to 25° C. By the action of the amylase, staling of starch can be prevented to maintain the texture of the food even after storage at low temperatures.

### <<Method of Stabilizing Amylase>>

The method of stabilizing an amylase of the present invention includes mixing an amylase, a polyhydric alcohol, and a saccharide containing a glucose unit. The amylase, the polyhydric alcohol, and the saccharide containing a glucose unit are as described above. The amylase, the polyhydric alcohol, and the saccharide containing a glucose unit may be mixed in any order.

### EXAMPLES

The present invention is described below with reference to examples, but the present invention is not limited to these examples. Hereinafter, “parts” and “%” mean “parts by weight” and “% by weight”, respectively, unless otherwise noted.

### (1) Materials Used

Soybean whey (Showa Sangyo Co., Ltd.)
Glycerol (Sakamoto Yakuhin Kogyo Co., Ltd., food additive glycerol RG)

### Dextrin (Matsutani Chemical Industry Co., Ltd., MAX 1000)

### Sorbitol (Ueno Fine Chemicals Industry, Ltd., SORBITOL-UENO 20M)

### Maltose (Hayashibara Co., Ltd., SUNMALT S)

### Maltitol (Fujifilm Wako Pure Chemical Corporation)

### Filter aid:

### Topco Perlite #54 (Toko Perlite Industry Co., Ltd.)

### KC FLOCK W-100 (Naigai Flour Milling Co., Ltd.)

### (2) Preparation of Amylase Composition

To soybean whey were added Topco Perlite #54 and KC FLOCK W-100 in a weight ratio of 0.3%, and the mixture was subjected to clarification and filtration with a filter press to obtain a filtrated sample which was then concentrated with an UF membrane (Daicen Membrane-Systems Ltd., FS10-FS-FUY03A1) to obtain a liquid soybean whey concentrate.

The soybean whey concentrate, polyhydric alcohols, and saccharides containing a glucose unit were mixed in the ratios shown in Tables 1 to 5. To each mixture was added a sodium hydroxide solution to adjust the pH to 5.2, followed by stirring for one hour. After the stirring, the samples were subjected to clarification and filtration using Topco Perlite and KC FLOCK W-100. The filtrated samples were subjected to bacteria elimination using a 0.2-μm filter (Toyo Roshi Kaisha, Ltd., C020A047A) to obtain amylase compositions.

### (3) Measurement of β-Amylase Activity

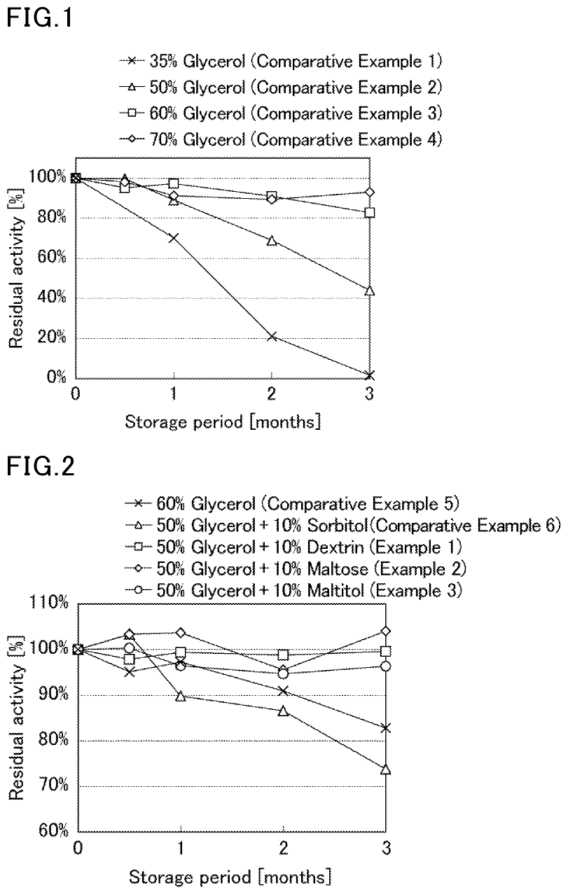

The activity of the amylase compositions was measured immediately after preparation and after storage at 40° C. for half a month, one month, two months, and three months as described below, and the relative activity was calculated relative to the activity immediately after preparation taken as 100%. The results are shown in FIGS. 1 to 5.

The β-amylase activity was tested by measuring a reducing sugar by a quantitative method with 3,5-dinitrosalicylic acid (DNS method). In the testing method, 1 ml of the amylase composition was added to 9 ml of a 1.1% glucose substrate solution containing a phosphate buffer with a pH of 5.5 and they were reacted at 60° C. for 30 minutes. After 30 minutes from the start of the reaction, 1 ml of the reaction solution was added to 3 ml of a DNS solution and then boiled for 15 minutes. After the boiling, the resulting solution was cooled to room temperature and then diluted with distilled water up to 25 ml in a measuring flask. The dilution was measured for absorbance at 550 nm. The concentration of the reduced glucose was determined from the absorbance using a calibration curve. One unit is defined as the amount of enzyme that produces 1 mg of maltose per hour under conditions including pH 5.5 and 60° C.

TABLE 1

| | Comparative Example | | | |
|---|---|---|---|---|
| | 1 | 2 | 3 | 4 |
| Glycerol | 35% | 50% | 60% | 70% |
| Soybean whey concentrate | 65% | 50% | 40% | 30% |

TABLE 2

| | Comparative Example | | Example | | |
|---|---|---|---|---|---|
| | 5 | 6 | 1 | 2 | 3 |
| Glycerol | 60% | 50% | 50% | 50% | 50% |
| Sorbitol | | 10% | | | |
| Dextrin | | | 10% | | |
| Maltose | | | | 10% | |
| Maltitol | | | | | 10% |
| Soybean whey concentrate | 40% | 40% | 40% | 40% | 40% |

TABLE 3

| | Comparative | | | | |
|---|---|---|---|---|---|
| | Example | Example | | | |
| | 7 | 4 | 5 | 6 | 7 |
| Glycerol | 50% | 50% | 50% | 50% | 50% |
| Dextrin | | 2.5% | 5% | 7.5% | 10% |
| Soybean whey concentrate | 50% | 47.5% | 45% | 42.5% | 40% |

TABLE 4

| | Comparative | | | | | | |
|---|---|---|---|---|---|---|---|
| | Example | | Example | | | | |
| | 7 | 8 | 8 | 9 | 10 | 11 | 12 |
| Glycerol | 50% | 40% | 40% | 40% | 40% | 40% | 40% |
| Dextrin | | | 2.5% | 5% | 7.5% | 10% | 12.5% |
| Soybean whey | 50% | 60% | 57.5% | 55% | 52.5% | 50% | 47.5% |
| concentrate | | | | | | | |

TABLE 5

| | Example | Comparative Example | | | | | |
|---|---|---|---|---|---|---|---|
| | 13 | 8 | 9 | 10 | 11 | 12 | 13 |
| Glycerol | 40% | 40% | | | | | |
| Dextrin | 10% | | 2.5% | 5% | 7.5% | 10% | 12.5% |
| Soybean whey | 50% | 60% | 97.5% | 95% | 92.5% | 90% | 87.5% |
| concentrate | | | | | | | |

As shown in FIG. 1, when the glycerol concentration was 35 to 50% (Comparative Examples 1 and 2), the enzyme stability at 40° C. was low and the relative activity was greatly lost after storage at 40° C. for three months. When the glycerol concentration was 60 to 70% (Comparative Examples 3 and 4), the relative activity was maintained due to the reduced water content, but a high concentration of the amylase in the composition was not achieved.

As shown in FIG. 2, the combined use of 50% of glycerol and 10% of a saccharide containing glucose as a constituent unit greatly improved the stability of the amylase (Examples 1 to 3). When 60% of glycerol alone (Comparative Example 5) or a combination of 50% of glycerol and 10% of sorbitol (Comparative Example 6) was used, the storage stability of the amylase was lower than that in the test examples containing a saccharide containing glucose as a constituent unit.

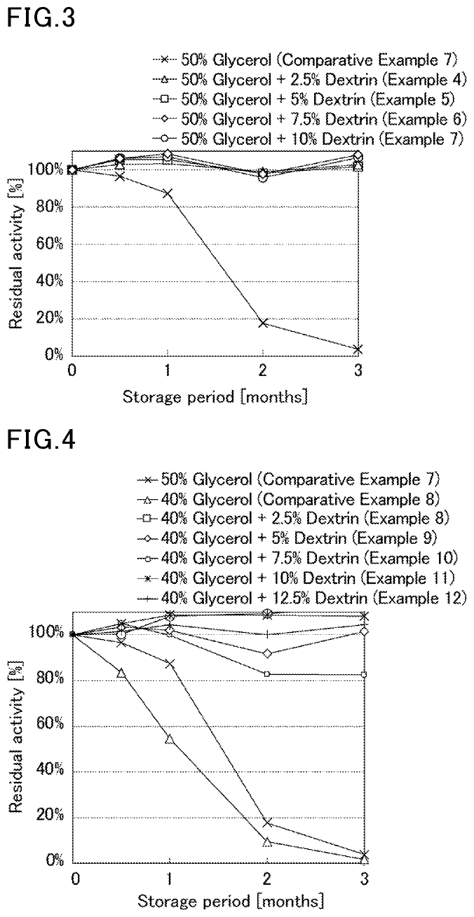

As shown in FIG. 3, the combined use of 50% of glycerol and 2.5 to 10% of a saccharide containing glucose as a constituent unit greatly improved the stability of the amylase (Examples 4 to 7).

As shown in FIG. 4, when 40% of glycerol was combined with 2.5 to 12.5% of a saccharide containing glucose as a constituent unit, a high concentration of the amylase in the composition was achieved and at the same time the stability of the amylase was improved (Examples 8 to 12).

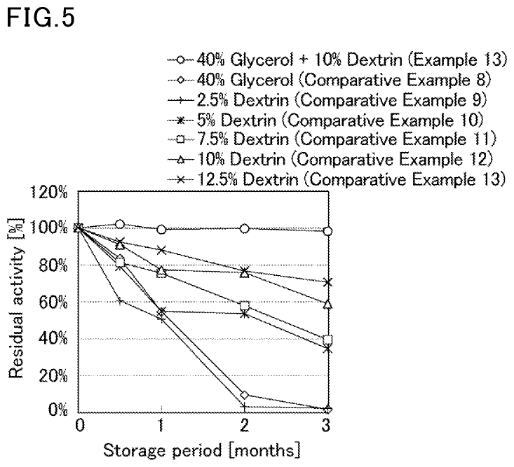

As shown in FIG. 5, when 40% of glycerol was combined with 10% of a saccharide containing glucose as a constituent unit, the stability of the amylase was improved (Example 13). When glycerol or a saccharide containing glucose as a constituent unit was used alone, the stability of the amylase was low (Comparative Examples 8 to 13).

### (4) Viable Bacteria Count in Amylase Composition

The viable bacteria count in the amylase compositions was determined immediately after preparation and after storage at 40° C. for half a month, one month, two months, and three months as described below. The results are shown in Table 6.

An amount of 25 ml of the amylase composition was weighed and diluted 10-fold with 225 ml of saline, and the dilution was well mixed. 10³ to 10⁶-fold dilution series were prepared from the 10-fold diluted amylase composition using a phosphate buffer. An amount of 1 ml of each dilution was placed in a 90-mm diameter plate, and 15 to 20 ml of a tryptone glucose yeast extract medium (46° C.±1° C.) was added and mixed to prepare plates. The viable bacteria count of each dilution was determined with n=2. The plates were incubated at 30° C.±1° C. for 72 hours and the number of colonies appearing on each plate was counted.

When the number of colonies on the plate with the highest dilution rate was greater than 300 CFU, only the plate with that dilution rate was counted. When the number of colonies was smaller than 30 CFU, only the plate with the lowest dilution rate was counted. When the number of colonies was 30 to 300 CFU, an average of the two plates having different dilution rates was calculated.

TABLE 6

| | | Storage period (months) | | | | |
|---|---|---|---|---|---|---|
| | | 0 | 0.5 | 1 | 2 | 3 |
| Example 13 | 40% Glycerol + | 20 | <10 | <10 | <10 | <10 |
| | 10% Dextrin | | | | | |
| Comparative | 40% Glycerol | 1670 | <10 | <10 | <10 | <10 |
| Example 8 | | | | | | |
| Comparative | 2.5% Dextrin | 6000 | >300,000 | 1,000,000 | 300,000 | 240,000 |
| Example 9 | | | | | | |
| Comparative | 5% Dextrin | 7100 | >300,000 | 1,000,000 | 2,700,000 | 95,000 |
| Example 10 | | | | | | |
| Comparative | 7.5% Dextrin | 8200 | >300,000 | 5,200,000 | 3,000,000 | 70,000 |
| Example 11 | | | | | | |
| Comparative | 10% Dextrin | 7600 | >300,000 | 100,000 | >300,000 | 500,000 |
| Example 12 | | | | | | |
| Comparative | 12.5% Dextrin | 7700 | >300,000 | 400,000 | >300,000 | 650,000 |
| Example 13 | | | | | | |

In Example 13, no bacterial growth was observed even after a lapse of three months. In Comparative Examples 9 to 13, bacterial growth was observed. In Comparative Example 8, no bacterial growth was observed although the stability of the amylase was low as shown in FIG. 5.

1. An amylase composition, comprising:
a saccharide containing glucose as a constituent unit;
a polyhydric alcohol; and
an amylase.

2. The amylase composition according to claim 1,
wherein a combined amount of the saccharide containing glucose as a constituent unit and the polyhydric alcohol is 70% by weight or less.

3. The amylase composition according to claim 1,
wherein the polyhydric alcohol is selected from the group consisting of glycerol, sorbitol, and propylene glycol.

4. The amylase composition according to claim 1,
wherein the saccharide is at least one selected from the group consisting of sucrose, trehalose, dextrin, maltose, and maltitol.

5. The amylase composition according to claim 1,
wherein the amylase is a β-amylase.

6. A food additive, comprising the amylase composition according to claim 1.

7. A food, comprising the food additive according to claim 6.

8. A method of stabilizing an amylase, the method comprising
mixing an amylase, a polyhydric alcohol, and a saccharide containing a glucose unit.
